(19) United States
(12) Patent Application Publication
(10) Pub. No.: US 2013/0155376 A1
(43) Pub. Date: Jun. 20, 2013

(54) VIDEO GAME TO MONITOR VISUAL FIELD LOSS IN GLAUCOMA

(71) Applicant: ICHECK HEALTH CONNECTION, INC., Portland, OR (US)

(72) Inventors: David Huang, Portland, OR (US); Hiroshi Ishikawa, Wexford, PA (US)

(73) Assignee: ICHECK HEALTH CONNECTION, INC., Portland, OR (US)

(21) Appl. No.: 13/720,182

(22) Filed: Dec. 19, 2012

Related U.S. Application Data
(60) Provisional application No. 61/578,054, filed on Dec. 20, 2011.

(51) Int. Cl. A61B 3/024

(52) U.S. Cl. CPC A61B 3/024
USPC 351/224; 351/246

(57) ABSTRACT

Systems and methods for providing a video game to map a test subject's peripheral vision comprising a moving fixation point that is actively confirmed by an action performed by the test subject and a test for the subject to locate a briefly presented visual stimulus. The video game is implemented on a hardware platform comprising a video display, a user input device, and a video camera. The camera is used to monitor ambient light level and the distance between the device and the eyes of the test subject. The game serves as a visual field test that produces a visual field map of the thresholds of visual perception of the subject's eye that may be compared with age-stratified normative data. The results may be transmitted to a health care professional by telecommunications means to facilitate the diagnosis and/or monitoring of glaucoma or other relevant eye diseases.

### BACKGROUND OF THE INVENTION

1. Field of the Invention

The present invention is directed generally to systems and methods for monitoring eye disorders, and more particularly to providing programs or video games for monitoring visual field loss for diagnosing glaucoma.

2. Description of the Related Art

Glaucoma is a leading cause of blindness worldwide. Glaucoma is a degeneration of the optic nerve associated with cupping of the optic nerve head (optic disc). Glaucoma is often associated with elevated intraocular pressure (IOP). However, the IOP is normal in a large minority of cases and therefore IOP alone is not an accurate means of diagnosing glaucoma. One time examination of the optic disc is usually not sufficient to diagnose glaucoma either, as there is a great variation in the degree of physiologic cupping among normal eyes. Glaucoma eventually damages vision, usually starting in the peripheral region. Therefore, visual field (VF) tests that cover a wide area of vision (for example, 48 degrees) are a standard for diagnosing glaucoma. Visual field testing is also called “perimetry” and automated testing is called automated perimetry. A single, standard VF test is poorly reliable, however, due to large test-retest variation. Therefore, several VF tests are generally required to establish an initial diagnosis of glaucoma or to show a worsening of glaucoma over time. Some drawbacks of standard visual field testing include:
1) Dedicated instruments installed at an eye specialist's clinic are needed. This prevents frequent repetition of the test to confirm glaucoma diagnosis or to monitor the progression of the disease.
2) The test requires fixation at a fixed spot for many minutes. This is unnatural, tiring, and often not achieved. Fixation loss is a common cause of unreliable tests.
3) Subject input consists of simple yes-or-no clicking of a button. Since the timing of the click can be affected by poor subject attention, this contributes toward higher false positive and false negative responses. It also requires long intervals to separate presentation of visual stimuli. This causes boredom and loss of attention. This also prevents frequent repetition of the test.
4) The visual stimuli are uninteresting. This causes boredom and loss of attention.
5) The auditory environment is quiet. This causes boredom and loss of attention.
6) There is no immediate feedback on how the subject is doing. This causes boredom and loss of attention.
7) The subject's head is held in a chin rest to maintain a fixed distance to the visual stimuli. This is uncomfortable over extended periods of time. This prevents frequent repetition of the test.
8) Newer modalities of the visual field test that may be more sensitive for glaucoma detection, such as short-wavelength automated perimetry and frequency-doubling technology, require special instrumentations.

### BRIEF DESCRIPTION OF THE SEVERAL VIEWS OF THE DRAWINGS

FIG. 1 illustrates a display, input device, and distance-monitoring camera features of an embodiment of the invention implemented using a tablet computer;

FIG. 2 illustrates the operation of an ambient light monitoring camera and a viewing stand according to an embodiment of the present invention;

FIG. 3A illustrates the operation of a distance adjustment process using video analysis of a pattern printed onto an eye occluder;

FIG. 3B illustrates an enlarged view of the eye occluder shown in FIG. 3A;

FIG. 3C illustrates the operation of a second distance adjustment process that utilizes a regularly-spaced vertical line overlay;

FIG. 4 is a block diagram illustrating the relationship between a computer according to an embodiment and its input and output devices;

FIG. 5 illustrates a first screen shot of a butterfly game in accordance with an embodiment;

FIG. 6 illustrates a second screen shot of the butterfly game in accordance with an embodiment;

FIG. 7 illustrates a third screen shot of the butterfly game in accordance with an embodiment;

FIG. 8 illustrates a fourth screen shot of the butterfly game in accordance with an embodiment;

FIG. 9 illustrates a fifth screen shot of the butterfly game in accordance with an embodiment;

FIG. 10 is a flowchart depicting an integrated visual field game cycle;

FIG. 11 depicts a visual field output from the visual field game;

FIG. 12 is a flow chart depicting a selection of stimulus presentation locations for one round of the visual field game;

FIG. 13 is a flow chart depicting a testing cycle used to establish the threshold of visual stimulus perception;

FIG. 14 illustrates a first screen shot of an Apache helicopter gunner game in accordance with an embodiment;

FIG. 15 illustrates a second screen shot of the Apache helicopter gunner game in accordance with an embodiment;

FIG. 16 illustrates a third screen shot of the Apache helicopter gunner game in accordance with an embodiment;

FIG. 17 illustrates a fourth screen shot of the Apache helicopter gunner game in accordance with an embodiment;

FIG. 18 illustrates a fifth screen shot of the Apache helicopter gunner game in accordance with an embodiment;

FIG. 19 illustrates a sixth screen shot of the Apache helicopter gunner game in accordance with an embodiment;

FIG. 20 illustrates a first screen shot of a “Chase the Dot” game in accordance with an embodiment;

FIG. 21 illustrates a second screen shot of the Chase the Dot game;

FIG. 22 illustrates a third screen shot of the Chase the Dot game;

FIG. 23 illustrates a fourth screen shot of the Chase the Dot game;

FIG. 24 illustrates a fifth screen shot of the Chase the Dot game;

FIG. 25 illustrates a sixth screen shot of the Chase the Dot game;

FIG. 26 illustrates a seventh screen shot of the Chase the Dot game;

FIG. 27 illustrates an eighth screen shot of the Chase the Dot game;

FIG. 28 illustrates a ninth screen shot of the Chase the Dot game;

FIG. 29 is a plot of reaction time distribution for suprathreshold and subthreshold visual stimuli;

FIG. 30 is a flow chart showing a reaction time-based visual field game cycle; and

FIG. 31 is a diagram of a hardware environment and an operating environment in which the computing devices of the systems disclosed herein may be implemented.

### DETAILED DESCRIPTION OF THE INVENTION

### Overview

Embodiments of the present invention are directed to a video game to map a test subject's peripheral vision. In some embodiments, the video game comprises a moving visual fixation point that is actively confirmed by an action performed by the test subject and a test for the subject to locate a briefly presented visual stimulus (e.g., 0.1 seconds, 1 second, etc.). The game is implemented on a hardware platform comprising a video display, a user input device, and a video camera. The camera is used to monitor ambient light level and the distance between the video display and the eyes of the test subject. The game serves as a visual field test that produces a map of the thresholds of visual perception of the subject's eye that may be compared with age-stratified normative data. The test is suitable to be administered by the subject (also referred to as player or user herein) with or without professional supervision. The results may be transmitted to a health care professional or other entities by telecommunications means to facilitate the diagnosis and/or monitoring of glaucoma or other relevant eye diseases.

### The Apparatus

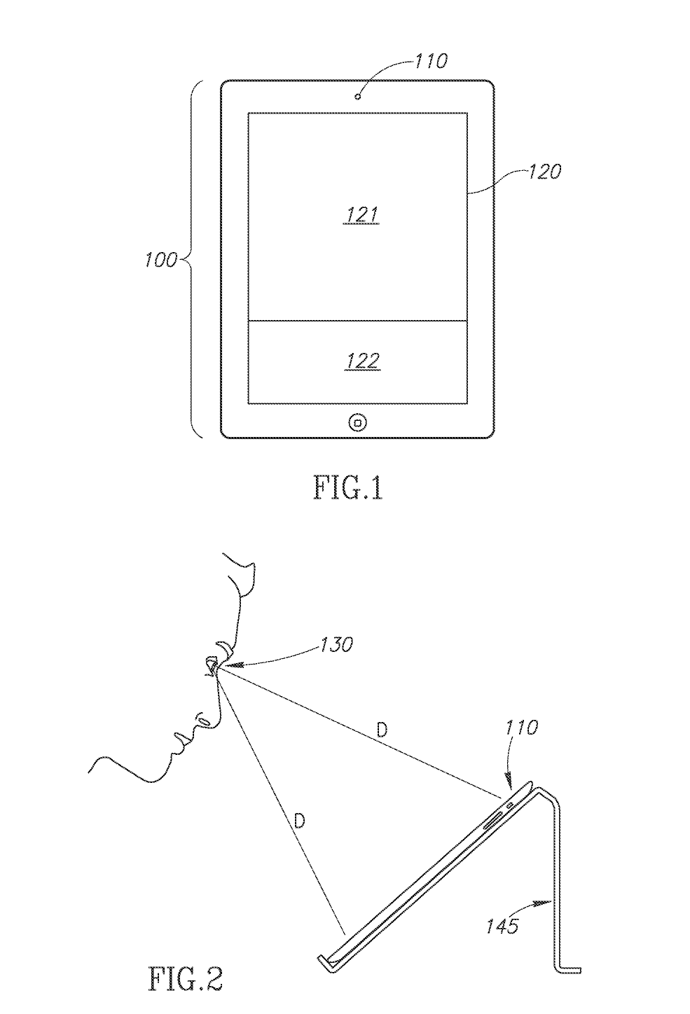

Embodiments of the present invention include a computer with a video display, a video camera, and a human-user input device. One example of an integrated apparatus serving these functions is the iPad 2® (Apple Inc., Cupertino, Calif.). Other computers or computer systems with similar functionalities may also be used. Referring to FIG. 1, a device 100 is shown that has a video camera 110 configured to monitor the distance between the device and a test subject's eyes. The device 100 also comprises a touch screen display 120 that is divided into a main game play area 121 and an ancillary area 122. The play area 121 is used to display the visual action of a game. The play area 121 is preferably approximately square, but other shapes may also be used. The ancillary area 122 is used to for ancillary human user input and score display, as discussed below. In other embodiments, the play area 121 and 122 may be combined or may be display alternately on the display 120.

Referring to FIG. 2, the device 100 may be positioned on a stand 145 such that the user's eye 130 is approximately equal distance (D) to the top and bottom of the device's display 120. The camera 110 on the front of the device 100 may be used to monitor ambient light. The test is preferably performed in dim room lighting (low scotopic). The brightness of the screen 120 may be automatically adjusted according to the ambient light level within an acceptable range. Outside of the acceptable range, a warning message on the screen 120 may be provided to instruct the user to increase or decrease the room lighting appropriately.

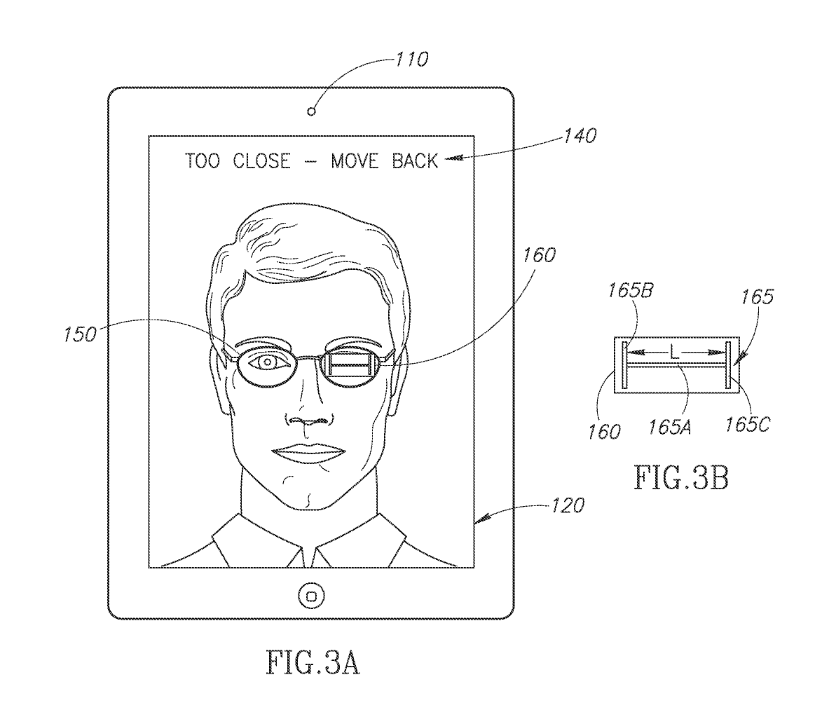

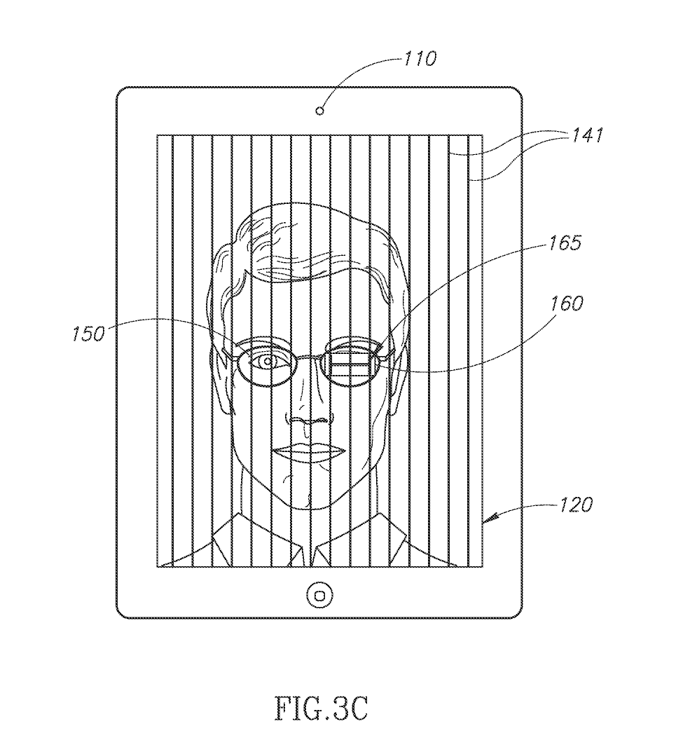

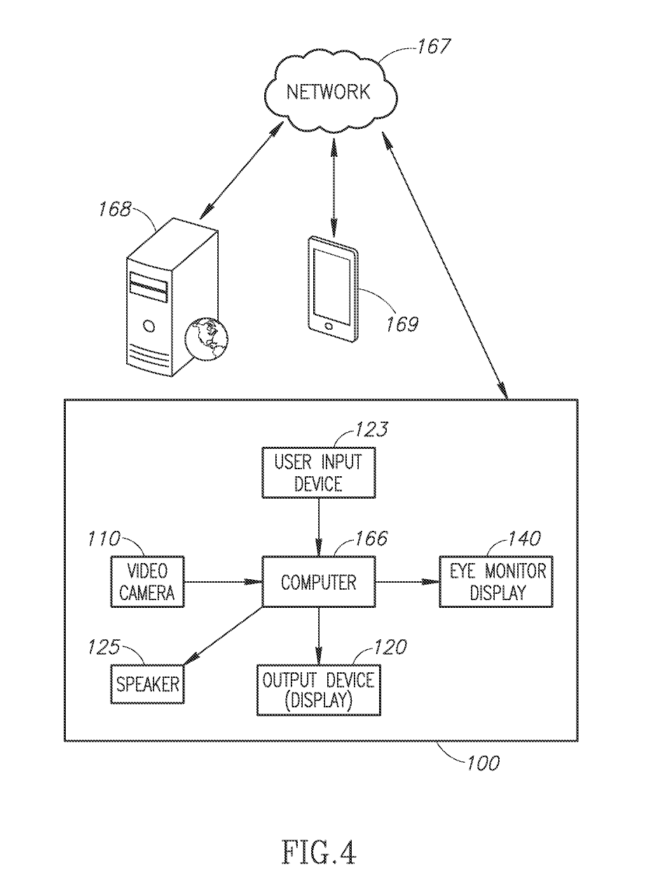

Referring to FIGS. 3A and 3B, an occluder 160 is shown that may be used to occlude vision in one eye so the other eye can be tested using the video game of the present invention. The occluder 160 could be mounted on spectacles 150 or could be fixed on the user's head using straps. The occluder 160 has a visible feature 165 of known dimensions which is captured by the video camera 110 and can be analyzed by a computer (see FIG. 4) of the device 100 to monitor the distance between subject's eyes and the device. As shown, the visual feature 165 could include, for example, a horizontal bar 165A with well-defined termination points (e.g., vertical bars 165B and 165C) so that the length of the horizontal bar may be easily determined by computerized automatic image processing. Other shapes or patterns, such as a circle or rectangle, could also be used. Based on the video analysis, the device 100 may display an instruction 140 on the screen 120 (and/or by sound) so the user can position his or her head within the optimal range of distance from the device.

An alternative method, shown in FIG. 3C, of obtaining the desired viewing distance D asks the user to adjust the viewing distance until the size of the real-time video display the occluder 160 has the correct size. In the example shown, the user compares the video display of the calibration feature 165 against a regularly spaced vertical line overlay 141. The user moves his/her head and/or the device 100 back and forth until the length of the feature 165 (e.g., between vertical bars 165B and 165C) spans two interval spacing between the vertical lines 141.

Another alternative method for the device 100 to monitor viewing distance is to analyze the size of the subject's eye (e.g., corneal width from limbus to limbus) being tested or other features on the subject's face. For this alternative to work, a video frame may first be taken when the user's face is at a known distance from the camera 110. As an example, the distance could initially be established using a measuring tape or ruler with a known length.

Referring now to FIG. 4, a user input device 123 and an output device 120 are shown connected to a computer 166 of the device 100. The term computer used in this instance refers to processors, memory, data/control bus, etc., as opposed to the peripheral input and output devices. The input and output functions can both be performed on the same touch screen, as depicted in FIG. 1. The video camera 110 produces image frames that are processed by the computer 166 to monitor the distance between the subject's eyes and the device 100. The subject produces action in the video game with the input device 123 and the game background and actions are displayed on the video display or output device 120. The game sounds are output on a speaker 125.

The test results may be transmitted or uploaded (e.g., wirelessly) to a server 168 over a network 167 (e.g., the Internet, a mobile communications network, etc.). This feature allows for the storage, tracking, review, and analysis of the test results over time to detect patterns, such as the deterioration of a patient's vision. The patient, his or her healthcare professionals, or others may access the data stored on the server 168 through a web browser or via a link to an electronic health record system of a healthcare facility. The test results data may be processed and presented in a manner that is useful for the patient and/or healthcare provider to analyze the results.

The server 168 may also be configured to provide notifications or alerts to the patient or their healthcare provider for any changes in vision that may require further attention or treatment. These alerts may be sent to a patient's and/or healthcare provider's electronic devices (e.g., the mobile phone 169, a computer, etc.) via email, SMS messages, voice messages, or any other suitable messaging system. For example, if a manual or automated analysis of the uploaded test results reveals that a patient's vision is deteriorating, the server 168 may automatically send a message to the patient and/or a healthcare provider to alert them of the change in condition. Thus, appropriate action or treatment may be provided.

### Initial Setup

The user is instructed to perform setup steps by the device 100 without the need of human professional instruction and supervision, though a human supervisor could be helpful to assure proper use.

The first time the subject is taking a test, the subject's identifying information and date of birth (or age) are entered into the computer 166 (e.g., using the input device 123). Based on this information, the computer 166 retrieves the age-stratified average VF (i.e., maps of visual stimulus perception threshold for right and left eyes) of a normal population to use as an initial estimate of the subject's current VF map.

For repeat tests, the subject enters his or her username so the computer 166 may retrieve recent VF results from local memory or from remote storage (e.g., the server 168). The average of recent VF maps obtained from previous tests may be used as initial estimates of the VF for the current test.

Since a game is used to perform the VF test, the terms “game” and “test” are used interchangeably herein. Further, the user of the device 100 is the subject of the VF test and the game player. Therefore, the terms “user,” “subject,” and “player” are also used interchangeably.

Before and/or during each game, the brightness of the screen 120 may be monitored and adjusted to the desired range by the use of camera 110 as described above. If the ambient light detected by the camera 110 is too high or low to be compensated for by adjusting the brightness, a message may be displayed on the display area 120 so the user can adjust the light level in the room. The test should generally be administered with the light level in the low scotopic range.

The test is administered at a viewing distance that is sufficient to provide useful glaucoma diagnostic information. For example, the iPad 2® used in some embodiments has a screen that is 5.8 inches wide. Referring back to FIG. 1, the display area 120 uses this full width of the screen. This provides a maximum perimetry testing area of +/−20 degrees (40 degrees full field width) at a viewing distance of 16 inches, using the methods of the current invention. The width and height of the VF testing is preferably no smaller than this, but could be smaller if desired. The device 100 monitors the viewing distance by taking images of the user's face (see FIG. 3) using the camera 110. The computer 166 (see FIG. 4) analyzes the visible feature 165 on the occluder 160 to compute the distance between the camera 110 and the occluder 160, which is approximately the same as the viewing distance. At the setup of each game, the device 100 instructs the user to move his or her head into position so the image of their face (in particular, the occluder 160) can be captured by the camera 110 and displayed in display area 120. The device 100 then instructs the user to move closer to or further from the display area 120 to bring the user's eyes into the target range of viewing distance. The initial target range may be 15 to 17 inches, for example. In some embodiments, the device 100 periodically monitors the viewing distance and instructs the user to move closer to or further from the display area 120 throughout the game. Again, within a certain range, the device 100 can scale the entire test to accommodate the given distance. In case of the testing area getting smaller than 20 degrees, a warning message may be displayed to notify the user about the situation, but if the user accepts the limitation, a game may begin. The results of such a situation may be modified accordingly, while clearly indicating the situation.

Generally, the user should be wearing spectacle correction for their best vision within the operating range of the viewing distance. For an emmetrope, a pair of reading glasses with power of +2.25 D to +2.50 D would be optimal for the viewing distance of 16 inches. If spectacles are used, the occluder 160 should be mounted over the spectacle lens over the eye not being tested. If no spectacles are needed or if the subject is using contact lenses, the occluder 160 could be mounted over plano glasses or strapped on as an eye patch.

### Game Playing and Visual Field Test Cycle

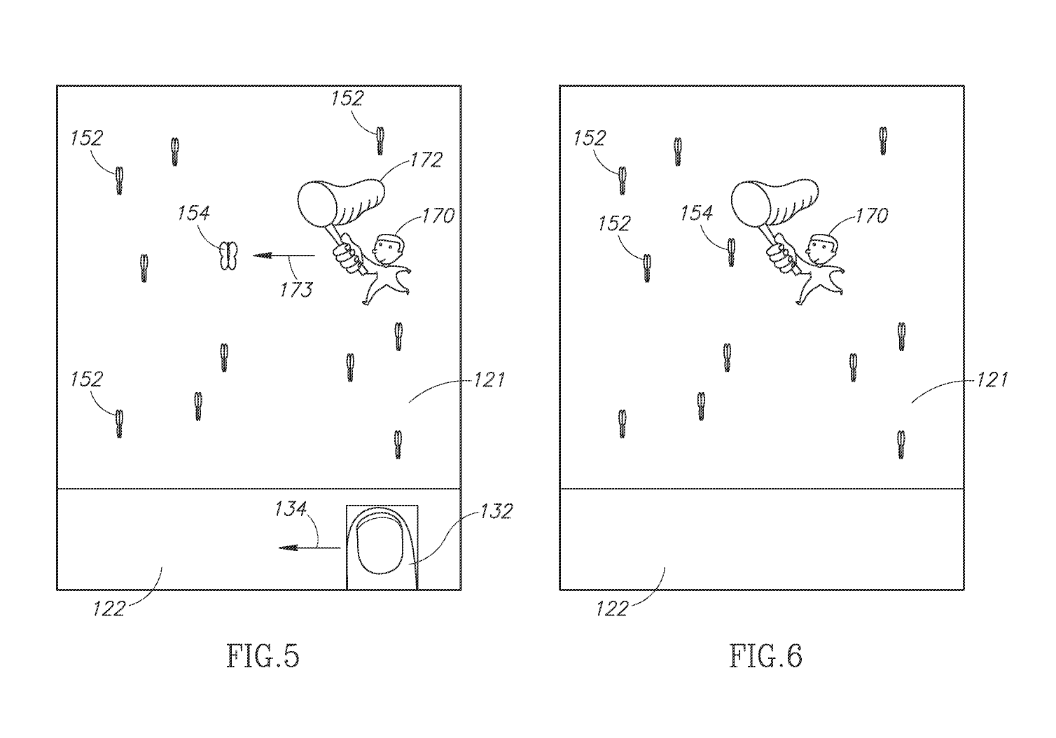

Many game scenarios could be devised based on the principles of the current invention. For the purpose of demonstration, a butterfly game is illustrated in FIGS. 5-9 and described below.

Referring to FIG. 5, the display area 121 has a field background (e.g., colored green) studded with many resting butterflies 152 with folded wings. The object of the game is to catch as many butterflies as possible when they take off and fly. Before taking off, a butterfly 154 slightly opens its wing for a brief moment. Seeing this signal, the game player (also the visual field test subject) swipes his (or her) finger 132 in the direction 134 so an action figure 170 with a net 172 moves in the same direction 173 to position a net 172 over the signaling butterfly 154.

Referring to FIG. 6, the butterfly 154 again folds its wings and rests after signaling. The user fine tunes the position of the net 172 by repeated small finger swipes to position the net over the butterfly 154 that has signaled. In an alternative embodiment, instead of swiping a finger in the ancillary area 122, the player directly taps on the butterfly 154 in the main display area 121 to position the net 172 over the butterfly.

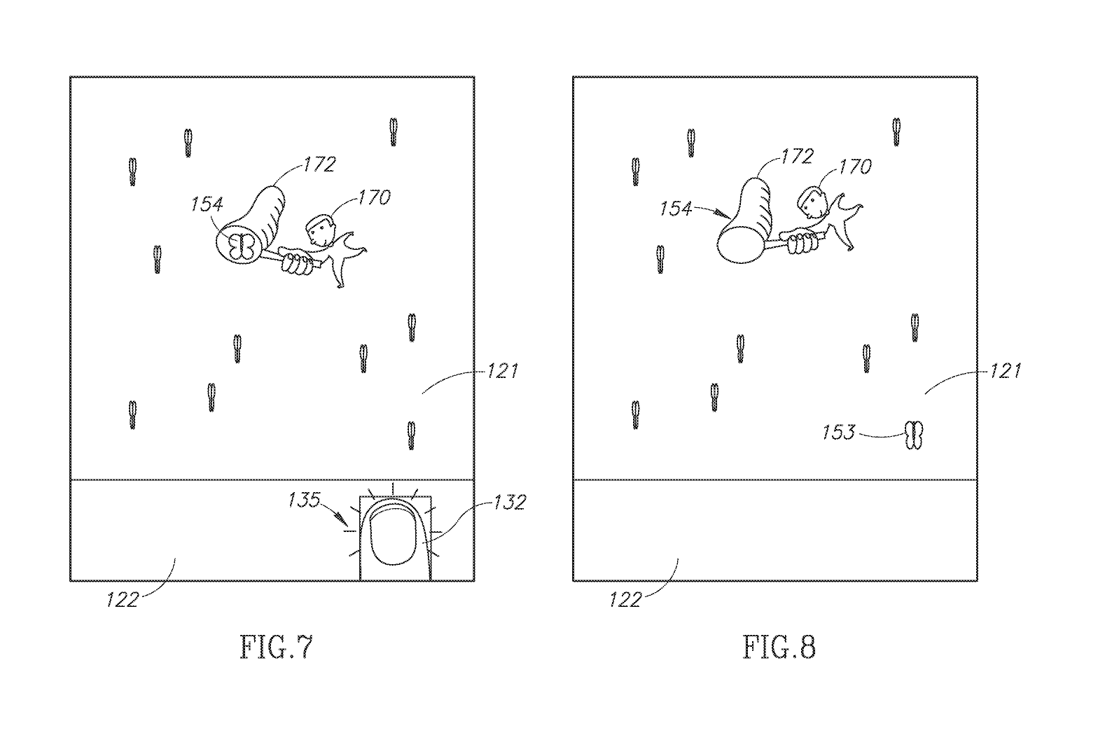

Referring to FIG. 7, the butterfly 154 that has previously signaled will, after some pause after signaling, begin to flap its wings vigorously for several seconds. To catch the butterfly 154, the user uses the finger 132 to perform a tapping action 135 which causes the net 172 to come over the butterfly 154 while it is lifting off (flapping its wings). The net 172 must close over the butterfly 154 at the right time and position to catch it. If not caught, the butterfly 154 would rapidly fly off the screen 121 or to another location on the screen.

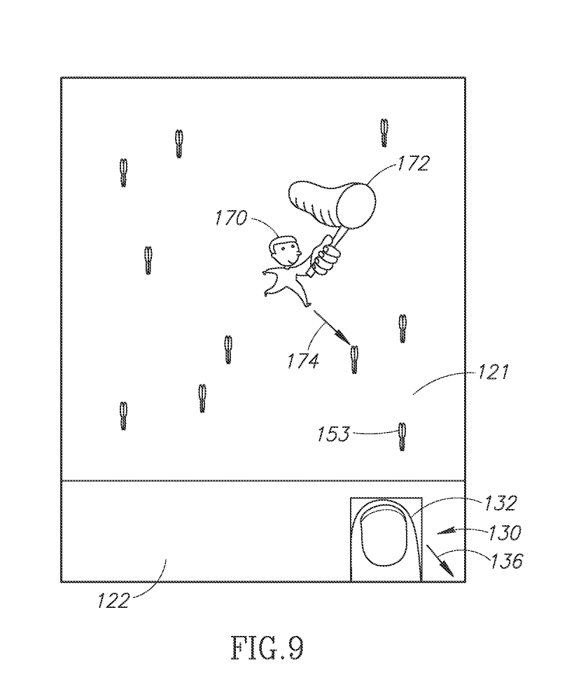

Referring to FIG. 8, when the net 172 catches the butterfly 154 (inside the net), the player's visual fixation is naturally still at the former position of butterfly 154. At this moment, another butterfly 153 produces a brief signal by opening its wings slightly and then closing them again. If the player sees this signal out of his peripheral vision, he would indicate that he saw the signal by moving the action figure 170 toward the butterfly 153. Referring to FIG. 9, the player swipes his or her finger 132 in the direction 136 so the action figure 170 moves in the direction 174 toward the butterfly 153 that has just signaled. This brings the game again to the beginning of the cycle.

For the user taking the test for the first time, the user's response time may be measured in the initial cycles (e.g., the initial five cycles) to establish the individual expected response time. A time window of the opened wings and the interval between cycles (independent from the user's success or false reaction) may be adjusted based on this measured response time.

The game cycle is continued with one of the butterflies 152 signaling and then flying off one at a time. When a preset number of butterflies 152 have been taken off the playing field (i.e., either caught or escaped), the game display area 120 (FIG. 1) is refreshed so a new arrangement of resting butterflies are placed thereon. Then, a new round of the game is played. The player is scored by the number of butterflies 152 caught per round. If the score is high enough for a sufficient number of rounds, then the game proceeds to a higher level where the butterflies 152 fly off more rapidly. This way, the game is kept at a sufficiently fast pace to keep the player's attention engaged. However, the difficulty level should be kept relatively low so the player captures a great majority of the butterflies 152. This helps provide good fixation and prevents frustration. Beside scoring and pacing, background music, action visuals, and sounds (e.g., butterfly fluttering in the net 172 with sound and an encouraging voice narrative) all may help to keep the player interested in the game.

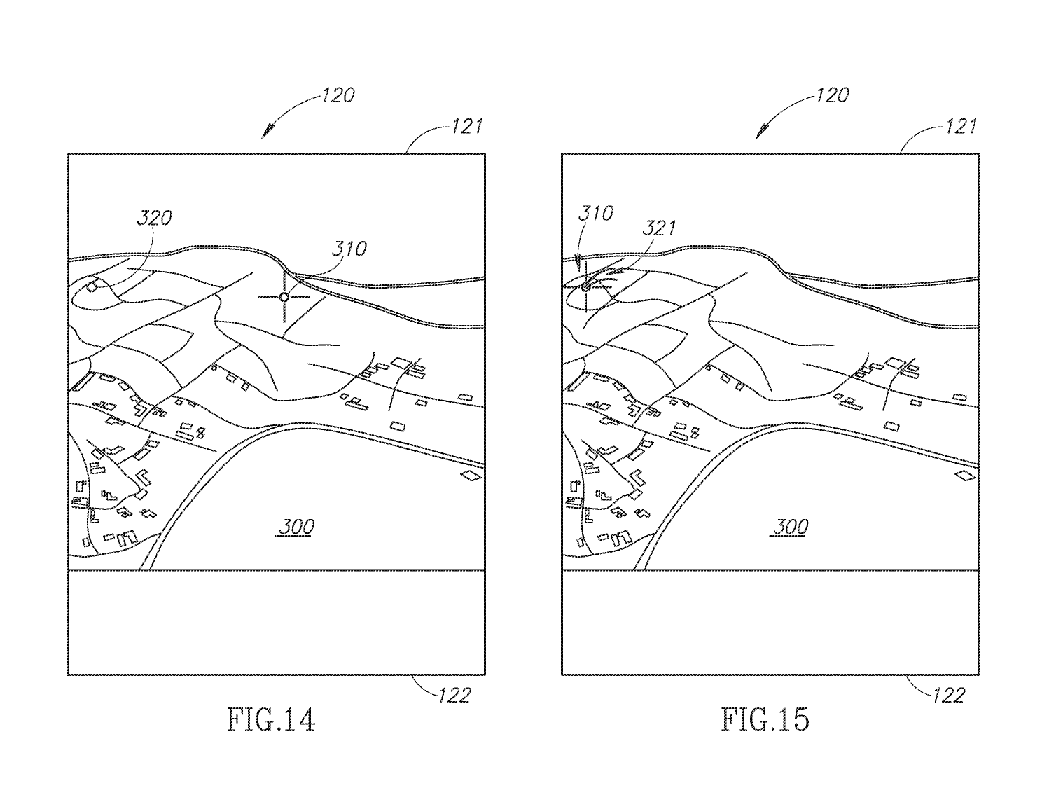

The butterfly game illustrated in FIGS. 5-9 is only one example of many possible scenarios. Other examples include catching frogs in a shallow pool, where the signal that serves as the visual field stimulus is ripples on the surface of the pool. It could also be a science fiction shooter game such as Star Trek®, where the goal is to shoot down enemy starships when they “decloak,” and the signal of a ship about to “decloak” is a ripple in a background star field, or the signal could be a brief flash in a dark background (see FIGS. 14-19). All of these games share common steps for establishing fixation, testing the visibility of a peripheral stimulus, and then a separate game task for the purpose of scoring and keeping the player engaged.

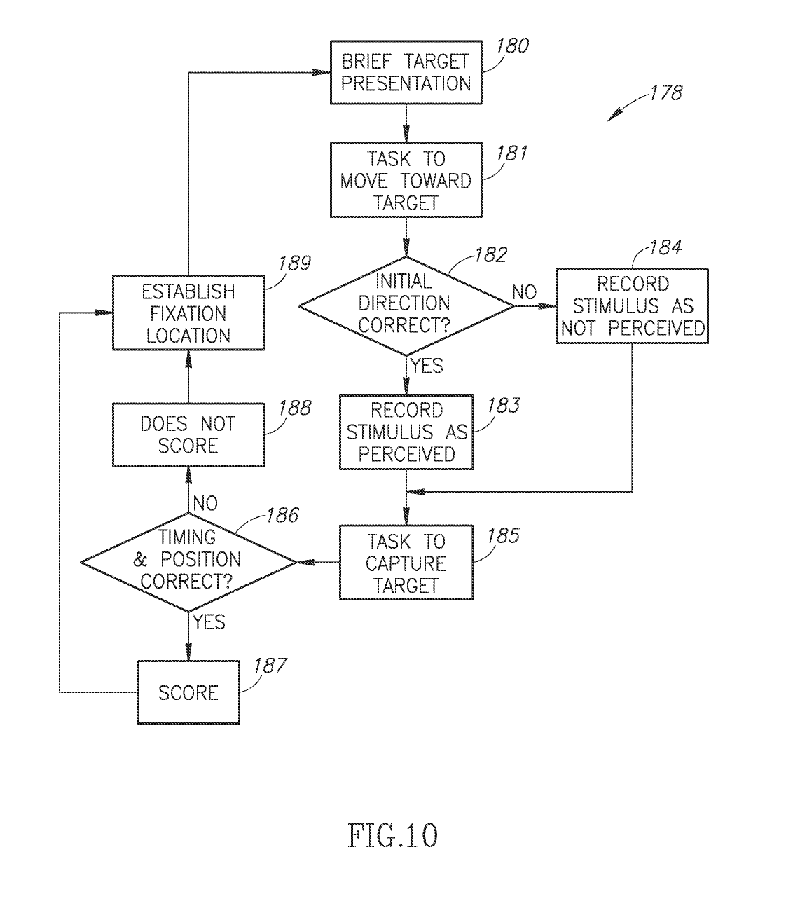

Referring to the process 178 shown in FIG. 10, a visual stimulus is briefly presented at a peripheral visual field location at 180. In a conventional static visual field, the visual stimulus is a brief presentation of a round target and the strength of the stimulus is determined by its size and brightness. The visual stimulus is conventionally white, or blue in the case of short wavelength automated perimetry. Motion is used in “frequency doubling technology.” The game visual field test of the present invention may use any combination of these visual stimulus design features. In the butterfly game illustrated in FIGS. 5-9, the brief opening of the butterfly wing is the visual signal or stimulus. In some embodiments, the opening exposes blue spots on the butterfly wings so there is a short-wavelength component to the stimulus. The opening may be a continuous motion so there also a motion component. The strength of the visual stimulus is determined by the width of wing opening, the length of the butterfly, and the duration of the wing opening and closing cycle. In conventional visual field testing, the subject clicks a button if he perceives the visual stimulus and takes no action if he does not.

In the game VF tests of the current invention, the subject is tasked to move the action symbol (i.e., the action figure 170 and net 172 of FIGS. 5-9) towards the visual stimulus in step 181 (FIG. 10). In this example, the subject indicates this direction by a finger swipe 136 on the ancillary area 122 of the touch screen 120 (FIG. 9). But this could also be accomplished using a touch pad, mouse, joystick, arrow keys, or other computer input device. If the initial direction entered by the subject is correct (FIG. 10, decision point 182 equals Yes), then it is very probable that the user has perceived the visual stimulus, and this is recorded at 183. If the initial direction entered by the subject is not correct, decision point 182 equals No, then it is probable that the user has not perceived the visual stimulus, and this is recorded at 184.

Referring still to FIG. 10, at 185 the player is tasked to capture the target. In the butterfly game shown in FIGS. 5-9 and discussed above, this means positioning the net 172 over the butterfly 154. Then the player must activate capturing (or shooting) of the target at the right time. In the butterfly game, this means tapping the input area 122 to cause the net 172 to come down when the butterfly 154 begins to fly off. If the timing and position of the net 172 is correct, decision point 186 equals Yes, then the butterfly is captured and the game score is increased at 187. Otherwise, the user does not score at 188. The scoring does not affect the VF test result, but serves to keep the player engaged. The target capture task also forces the subject's visual fixation on the capture target at 189, setting up the presentation of the next peripheral visual stimulus at 180. This brings the VF testing cycle back to the beginning.

### Monitoring of Eye Distance

At regular intervals during the game play and VF testing, the distance D between the subject's eyes and the device display screen may be monitored by analysis of video frames of the player's face (FIG. 3) as described for the beginning of the game. In some embodiments, this may be done between active game play intervals when the computer processor can analyze video frames without slowing down game play. The distance check may be done in the background without the player's knowledge. If the eye-to-display distance is within specified range, then no signal is given. If the eye-to-display distance is outside this range, then the video of the player's face may be displayed and instructions given to move further from or closer to the display to get within an optimal range. This procedure ensures that the peripheral visual field stimulus remains true to the specified visual angles. Alternatively, the system may scale the entire game according to the measured distance as described above. This feature is provided as an optional setup, which can be toggled on/off before starting a game by accessing a preference configuration pane.

In some embodiments, another check on working distance is achieved by intentionally placing a stimulus in the subject eye's blind spot. If the player detects the stimulus then the working distance may not be correct, or the player is not fixating properly. These fixation/position errors are recorded as a metric for the reliability of the test results.

### Mapping of Stimulus Perception Threshold

Referring to FIG. 11, in some embodiments, the output of the game VF test is a VF map 200 of the thresholds for perceiving the visual stimulus. The dimension of the map is limited by the size of the display 120 and the viewing distance D. For example, the iPad 2® has a display area that is 5.8 inches wide. This provides a maximum visual field width of +/−20 degrees (40 degrees full field width) at a viewing distance of 16 inches. In the example shown in FIG. 11, the 40×40 degree field is divided into 5×5 degree blocks to yield an 8×8 grid of visual stimulus presentation locations. The VF map 200 is presented as a grid of squares 205 labeled with sensitivity values. Sensitivity is the inverse of the minimum stimulus strength needed for the eye to perceive the stimulus at the particular location in the user's VF. The strength of the stimulus is specified as a combination of the size, brightness (in contrast to the background), and duration of the stimulus. For the butterfly game, the brightness may be held constant and the stimulus strength may be determined by the length of the butterfly, the width of opening, and the duration of the wing-opening signal. For other games, the stimulus strength could include variations in brightness and contrast as well. These parameters may be described on a logarithmic scale relative to a standard reference. The standard unit of the logarithmic scale is decibel (dB). The standard reference (i.e., 0 dB) can be set arbitrarily at first, and then calibrated to the perception threshold of the normal population.

In FIG. 11, the numbers in the squares 205 of the VF map 200 are dB sensitivity values relative to the average of the normal population (normative reference). A center point 201 represents the fixation point, corresponding to the foveal center anatomically. In this example of the VF of the right eye, the blind spot 202, corresponding to the optic nerve head anatomically, is to the right of and slightly inferior to the fixation point 201. The VF map format of the left eye is the mirror image. Four squares 203 around the blind spot 202 are not tested. Thus, there remain 60 squares 205 to be tested in the VF game. Glaucoma damages ganglion cells in the retina so the perception threshold goes up (sensitivity goes down). Areas of glaucoma damage 204 can be detected as clusters of decreased sensitivity that appear reliably on repeat testings.

The VF map 200 is mapped over several rounds of the VF game. The distribution of visual stimulation targets (e.g., butterflies) on the game display may be chosen randomly at each round of the game so no two rounds are likely to be the same. This keeps the game interesting. Predetermined patterns may also be used if desired (e.g., to ensure the data needed to generate the VF map 200 is obtained). For the butterfly game, the visual stimulation targets are the resting butterflies on the field (see FIG. 5). To generate the distribution of targets, in some embodiments a random selection algorithm (see FIG. 11) is applied to a map of VF testing locations.

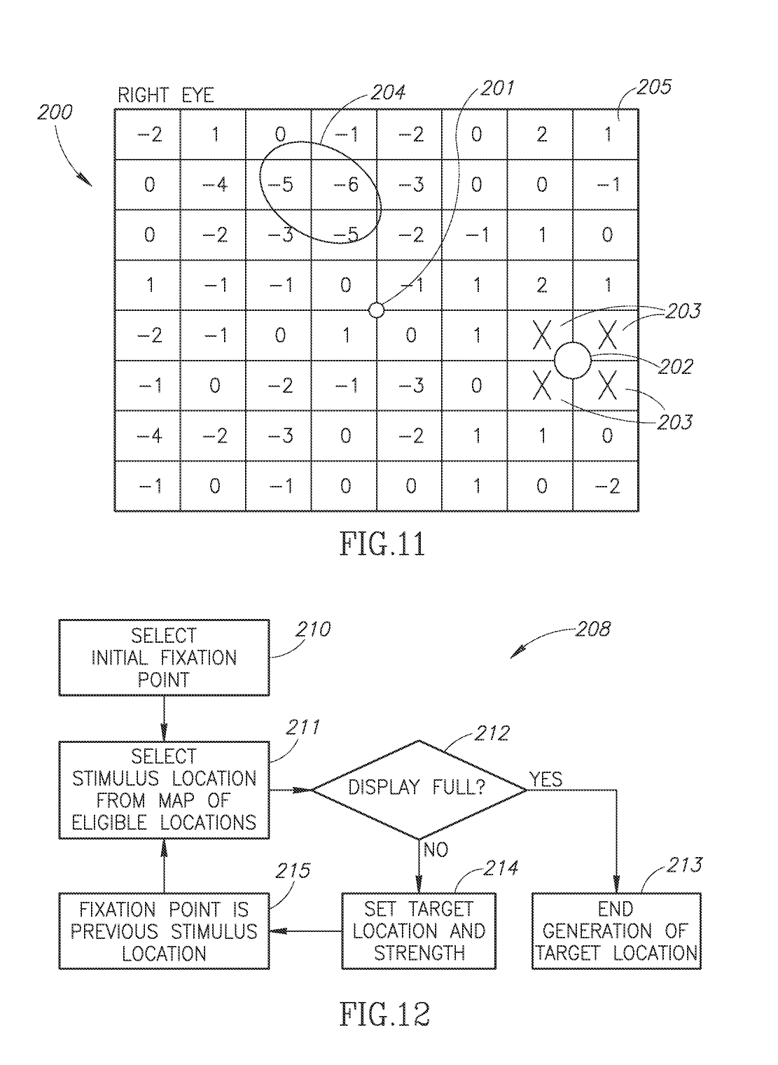

Referring to a process 208 shown in FIG. 12, one location on the display 120 is chosen (e.g., randomly) to be the initial location of the fixation point at 210. The location of the first peripheral visual stimulus is then selected (e.g., randomly) at 211 from the eligible locations constrained by the display area 120 and map of test locations yet to be measured. The probability of selecting a location is preferably proportional to the difference between the upper and lower bounds of the estimate of the perception threshold. If the display is full of VF targets, decision point 212 equals Yes, then no more target generation is needed at 213. Otherwise, the target setting process is continued. The target display location is determined by the display location of the fixation point and the VF location at 214. These are specified in degrees of visual angle. For example, if the display location (x, y) of the fixation point is (−2.5, +12.5) and the VF location is (7.5, −7.5), then the display location of the target is their sum (+5.0, +5.0). The stimulus strength is set according to an algorithm described below. Once a target is presented, it becomes the fixation point for the presentation of the next target at 215. The location of the next target stimulus is then selected, repeating step 211, and set relative to the fixation point. This completes the target selection cycle.

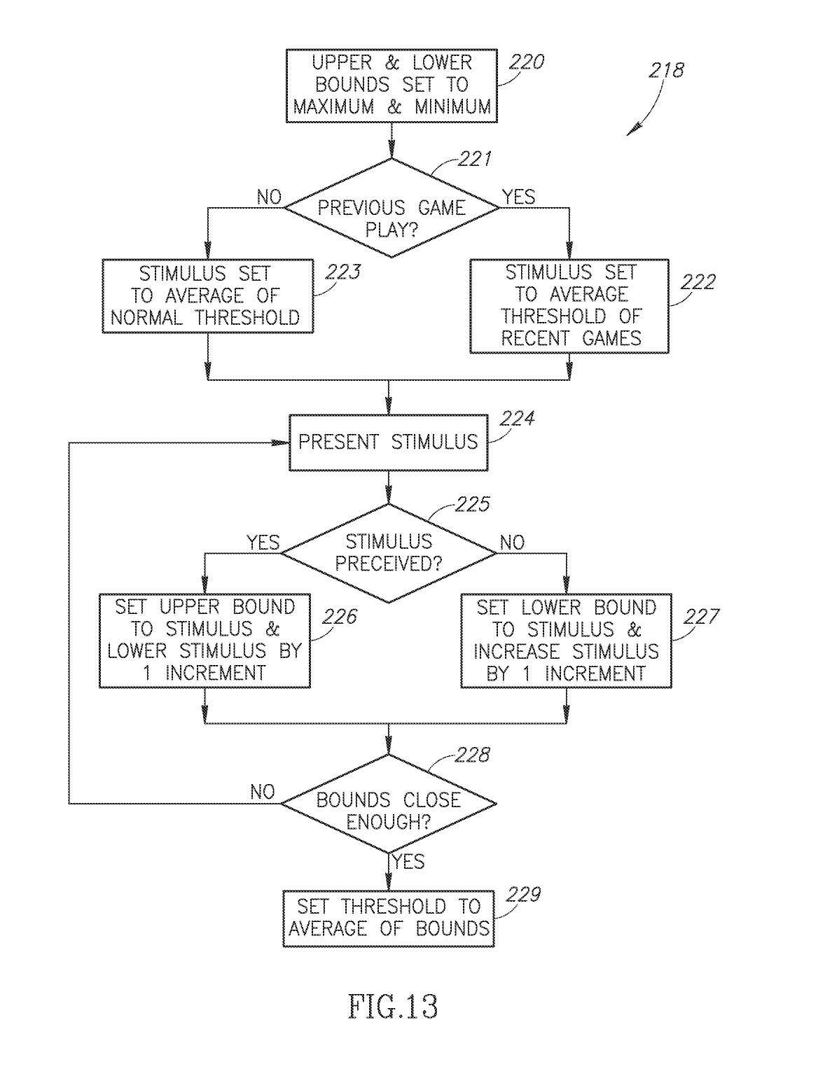

Referring now to the process 218 shown in FIG. 13, at the beginning of the game, the perception threshold of the user is unknown and therefore the upper and lower bounds are set to the maximum and minimum possible stimulus strengths, respectively, at 220. The initial strength of the stimulus at a particular VF location is set depending on whether there are any previous results for the eye being tested, decision point 221. If there had been previous VF tests, decision point 221 equals YES, the initial stimulus is set to the average result of the most recent three tests within the previous six months at 222. If fewer than three tests were done in the past six months, then the available tests are averaged. If the last game was more than six month ago, then the most recent test result is used. If this is the first test for the eye, then the initial stimulus strength is set to the average result of the normal population at 223. Other methods may be used to set the initial strengths of the stimulus.

Once the initial values are set, the VF testing cycle can begin. The stimulus is presented at 224. If the stimulus is perceived, decision 225 equals YES, then the upper bound is set to the level of the perceived stimulus and the next stimulus is set one increment lower at 226. The increment of adjustment is preferably approximately equal to the standard deviation of repeat testing. If the stimulus is not perceived, decision 225 equals NO, then the lower bound is set to the level of the stimulus and the next stimulus is set 1 increment higher in step 227. If the upper and lower bound are equal to or less than 1 increment apart, then the threshold can be calculated by averaging the upper and lower bounds at 228 and 229. If the bounds are more than 1 increment apart, then the testing continues. The VF test is continued until the threshold value has been determined at all locations. Other methods for approaching and determining the threshold value may be used. For example, rather than incrementing or decrementing the stimulus by 1 increment each interval, the stimulus may be set half way between the upper bound and lower bound at each interval.

Since any VF test is susceptible to error due to variation in the subject's response and loss of fixation from time to time, it is best to make diagnosis of glaucoma based on several VF tests. Likewise, worsening of the VF over time is best confirmed over several VF tests performed over a period of time. The advantage of the game VF test is that it is not as tedious and boring as conventional VF tests and therefore repeat testing is better tolerated. It can also be performed by users at home so that testing can be done continually between visits to a physician.

### Head Tracking and Gaze Tracking Game

The computing power of video game playing stations and mobile computing devices is increasing rapidly, such that real-time tracking of head position is possible by monitoring the position of gross facial features. It is also possible to monitoring fine eye features to determine the direction of gaze, or at least detect directional change in gaze. Using head position or gaze direction as input can speed up the input for VF games, compared to the use of manual input device such as finger swipe on the touch screen or joystick. Again, many scenarios are possible for such a game, but an “Apache gunner” game scenario is described herein and shown in FIGS. 14-19 as an example.

Referring to FIG. 14, a dark, low contrast background 300 is used. It depicts an aerial view of a town at night. A gun sight 310 is displayed on the display 120 (see FIG. 1) that follows the position of the player's head (or eyes), simulating a helmet-mounted gun sight worn by a gunner on an Apache attack helicopter. A calibrated flash 320 is presented as a VF stimulus, representing ground fire from the town. The player's task is to move the gun sight 310 to the target 320 by head motion (or eye motion). Since it is natural for a human to move his or her head and eyes toward a target, this instinctive movement make the game play more natural and rapid. For the purpose of VF testing, the computer measures the direction and timing of the player's head movement. If the movement is approximately towards the target 320 and within a specified time window, then the game determines that the subject has seen the peripheral visual target. The position of the target 320 relative to the fixation point 310 gives the VF location tested in terms of visual angle. The brightness and size of the flash 320 is used to test the perception threshold at the visual field location.

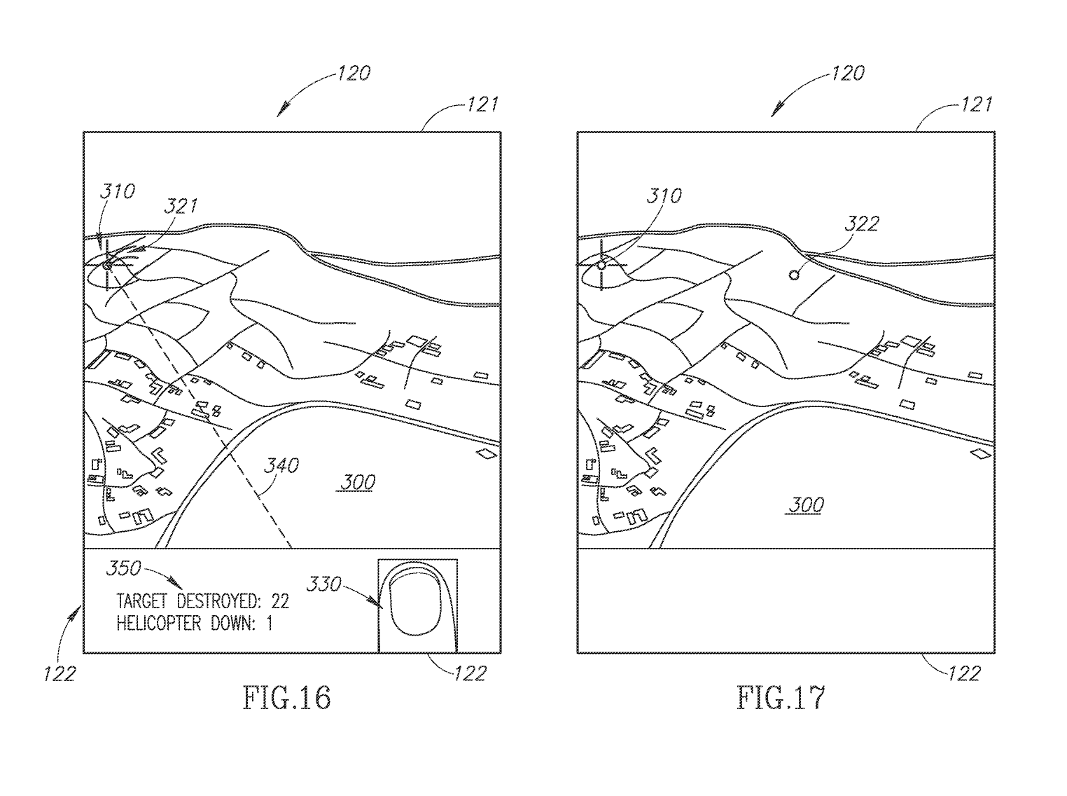

Referring to FIG. 15, the player moves the gun sight 310 so it is centered on the origin of ground anti-aircraft fire 321 displayed on the display 120. Referring to FIG. 16, the player taps a finger 330 on the touch screen within the ancillary area 122 in order to fire a machine cannon 340 onto the position of the gun sight 310, which is trained on the source of the anti-aircraft fire 321. The player must keep firing until the anti-aircraft fire 321 is silenced, or it is possible for the helicopter to be hit. If the helicopter is hit and grounded then the player obtains a new helicopter to play on. A game score 350 is kept based on the number of ground targets destroyed relative to the number of helicopters downed. The game score 350 is a goal to keep the player engaged and not strictly related to the VF stimulus perception threshold map. Thus, the video game and VF test are being carried out in parallel, but scores for each are kept separately.

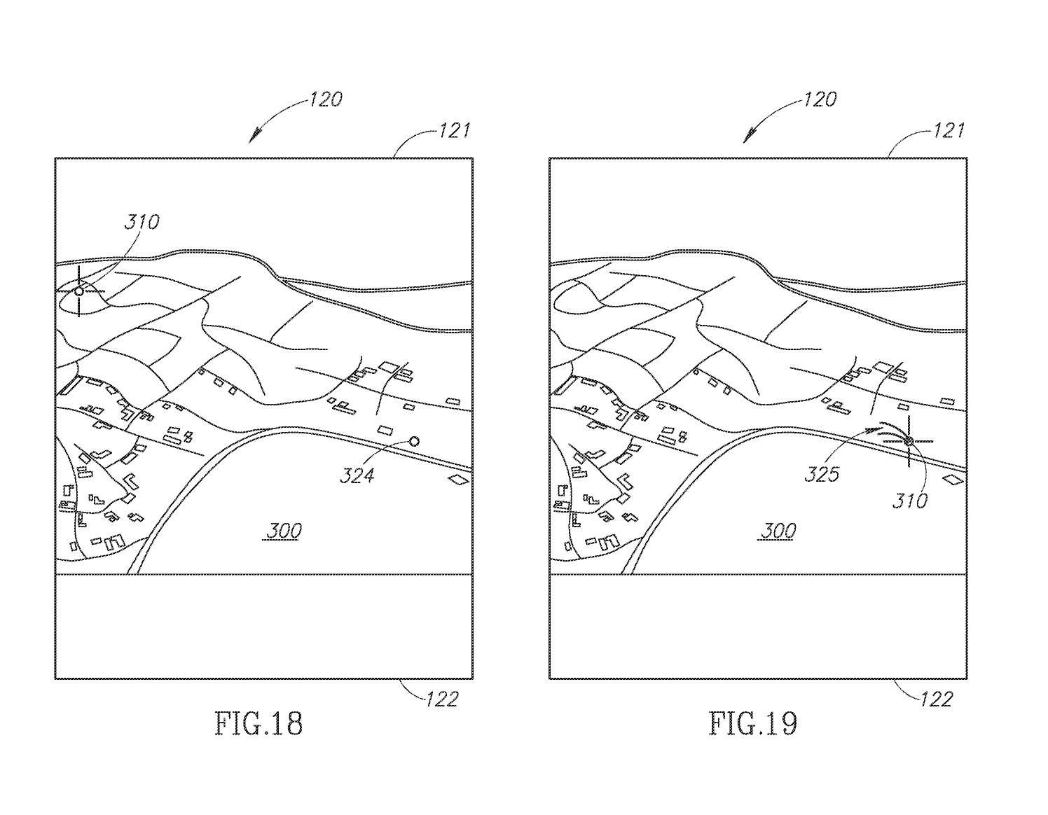

Referring to FIG. 17, after the ground target is destroyed, the crosshair position of the gun sight 310 becomes the new fixation position or point. A new VF test location is chosen and at that location, a flash 322 is presented briefly to test visual perception. In this instance, the subject did not perceive the new target 322 and there is no head movement toward the target within the specified time window. After the appropriate time delay, a new test location is chosen and a new flash 324 is presented there (FIG. 18). If the player sees the flash and moves the gun sight 310 toward it (FIG. 19), then the game cycle continues with the contest between the Apache helicopter and ground anti-aircraft fire 325 fired by ground gunners intent on destroying the Apache helicopter.

This game's scenario can also be played using a finger swipe on the touch screen 120 to control the gun sight 310 (or other manual control), instead of using head tracking. It can also be played using eye tracking to control the position of the gun sight 310. Whatever input device is used, it may be important for the main screen display area 121 to be kept clear of the player's finger and hand so as not to obscure the visual stimulus being displayed.

### Touch Screen Speed Tapping Game

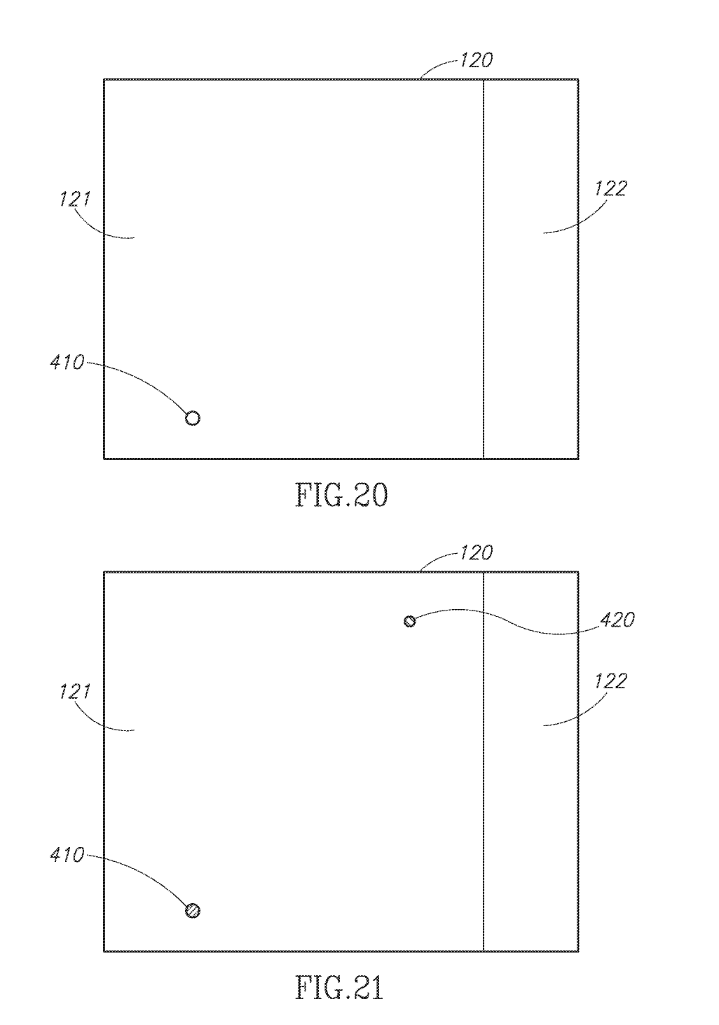

In yet another embodiment of the current invention, a game is optimized for speed on a touch screen tablet computer. Referring to FIG. 20, the user is instructed to look at white circle fixation point 410, which can be positioned anywhere on the game area 121 of the screen 120, including the edge of the screen. The game area 121 is preferably at a medium gray value. Referring to FIG. 21, the fixation point 410 flashes to attract player attention. At the same time, a peripheral stimulus 420 (e.g., a gray solid circle) appears on the display are 121 for a fraction of a second. The contrast (difference in brightness between the stimulus 420 and background 121), size, and duration of the circle define the stimulus strength. In some embodiments, the presentation duration is held constant and the contrast is varied. In some embodiments, the size of the stimulus 420 is only varied if the stimulus is not perceived even at maximum contrast.

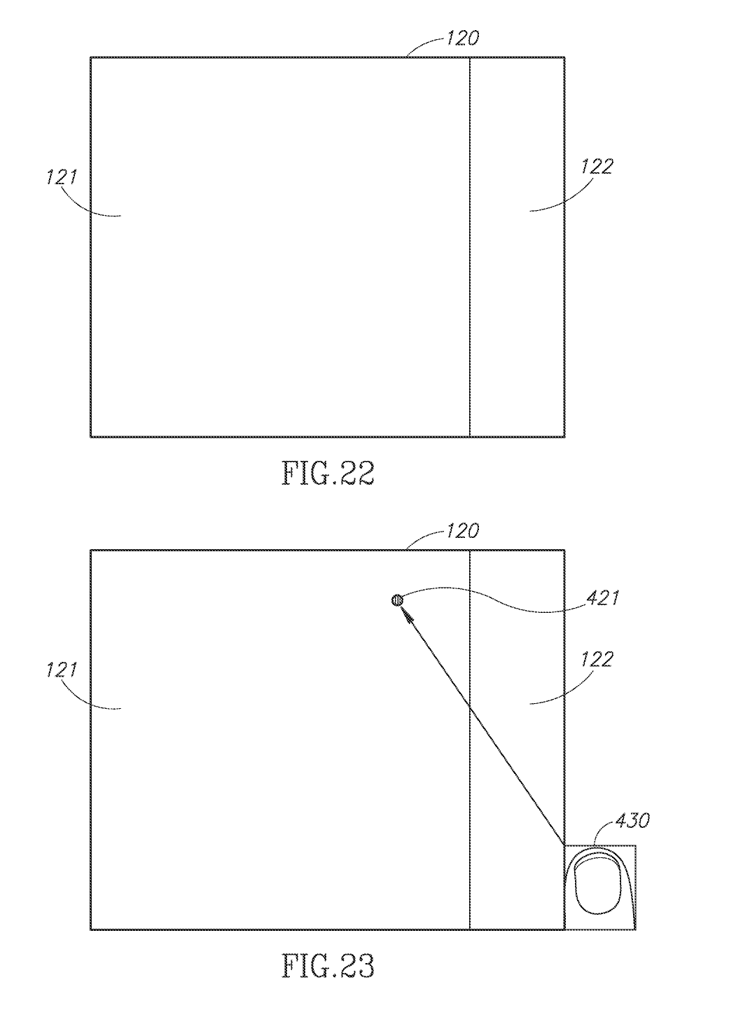

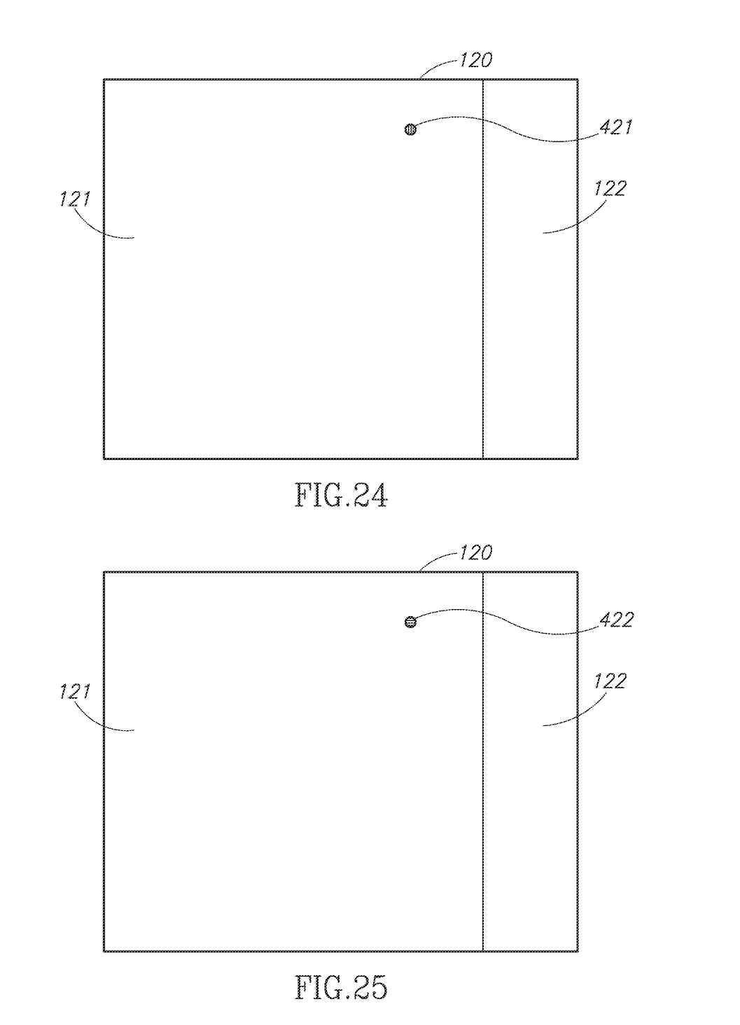

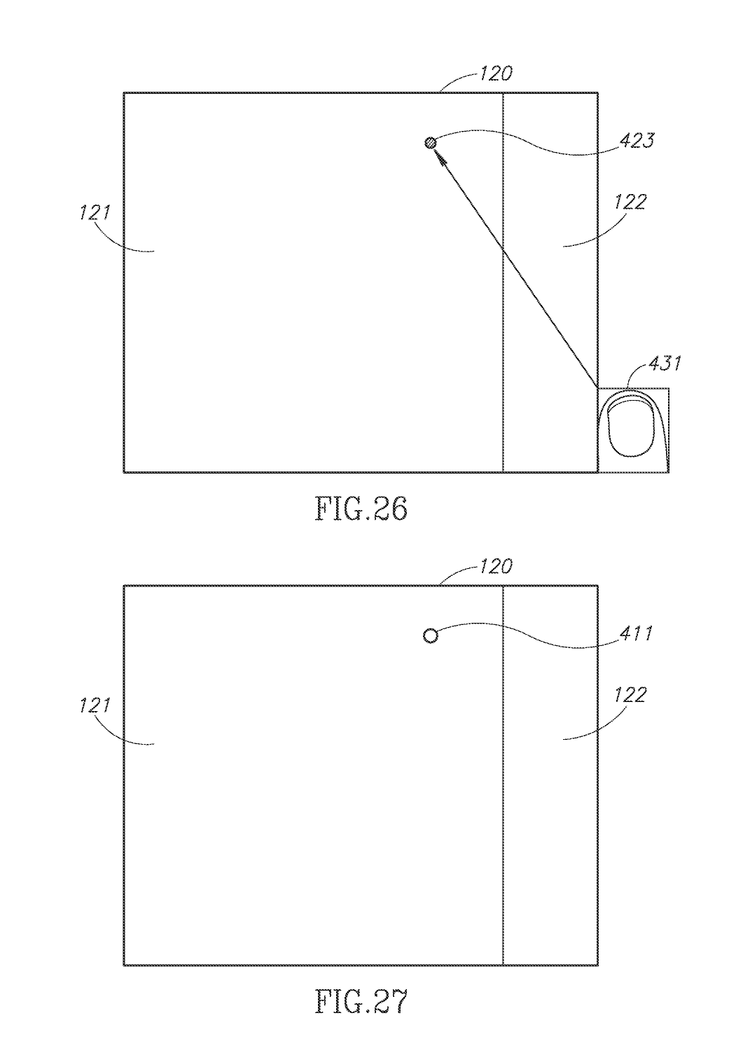

Referring to FIG. 22, both the fixation point 410 and the stimulus 420 disappear for a brief interval T1. The interval T1 could be a fraction of a second to a few seconds and is adjusted for optimal testing relative to the subject's reaction time. Referring to FIG. 23, after interval T1, a red target 421 (indicated by hatching) appears where the stimulus 420 was previously presented (see FIG. 21). If the player noticed the stimulus 420 before, he would be able to finger tap 430 on the target 421 rapidly and capture the red target. If the player did not perceive the stimulus 420 before, then the time needed for him to find and tap on the target 421 would be longer. Thus the reaction time R between the appearance of the target 421 and the finger tap 430 may be used to determine whether the stimulus 420 was perceived or not. Referring to FIGS. 24-26, if the player fails to tap on the red target 421 quickly, then the red target 421 turns sequentially into a green target 422 (FIG. 25) and a blue target 423 (FIG. 26), after interval times T2 and T3, respectively. Referring to FIG. 26, if the player finger tap 431 on the blue target 423 at this later stage, then he captures the blue target 423 instead of the red target 421. Referring to FIG. 27, the location of these targets becomes a new fixation point 411, and the game cycle begins again. In each cycle, a stimulus strength is tested at a visual field location, until the threshold stimulus strength is determined at all the visual field points as described above with reference to FIGS. 11-13.

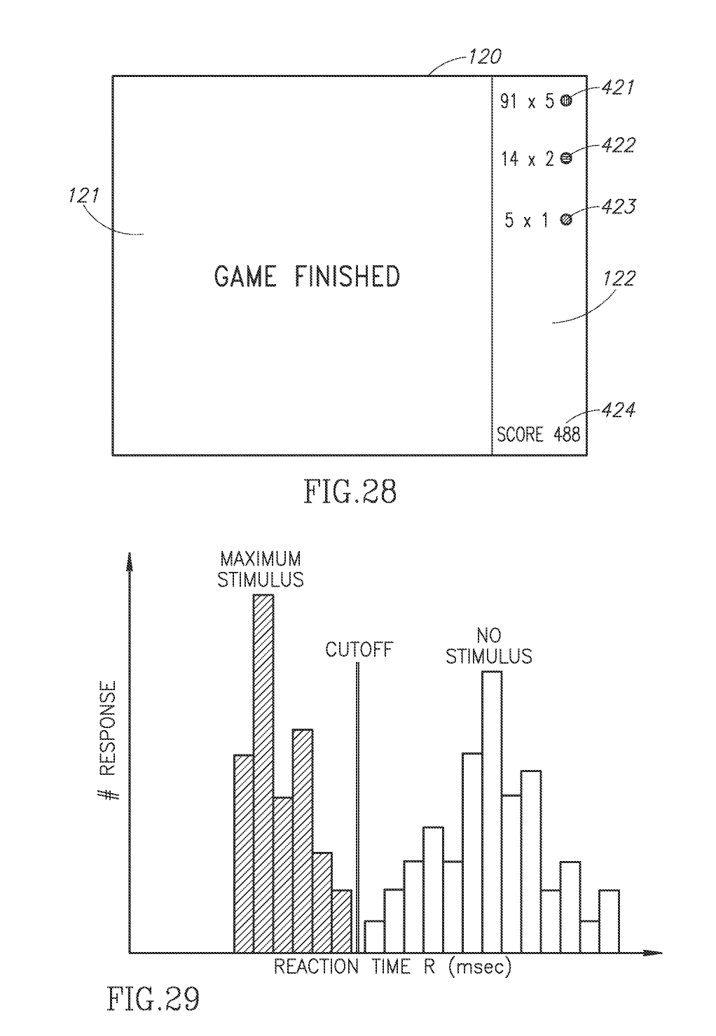

Referring to FIG. 28, at the end of the game (which is also the end of the visual field test), a game score 424 is tallied and provided in the ancillary area 122. The values of the captured targets are summed. Red targets 421 are worth more (e.g., 5 points) than green targets 422 (e.g., 2 points), which are in turn worth more than blue targets 423 (e.g., 1 point). The scoring motivates the player to tap as rapidly and accurately as he is able. This speeds up the testing process.

A potential drawback of this game is that the player's hand could potentially block his view of the game area 121. Therefore, the instructions for the game may advise the player to withdraw the hand after each tap so it does not block the view of the screen. Also, to ensure the user has moved his/her finger away, the game will wait until the detected touch is completely lifted off before moving to the next cycle.

Referring to FIG. 29, the reaction time R may be used to gauge whether a target is perceived or not. In order to calibrate the optimal cutoff time C, a calibration game may be played before any visual field testing is done. In the calibration game, the stimulus is either set at the maximum strength or set to zero strength (no stimulus). The cutoff time C is then set to optimize the discrimination between the two stimulus conditions. The time delays T1, T2, and T3 are also set in this process to be commensurate with the reaction time R. For long term monitoring of visual field, the reaction time is preferably calibrated on a regular basis to accommodate learning and aging effects.

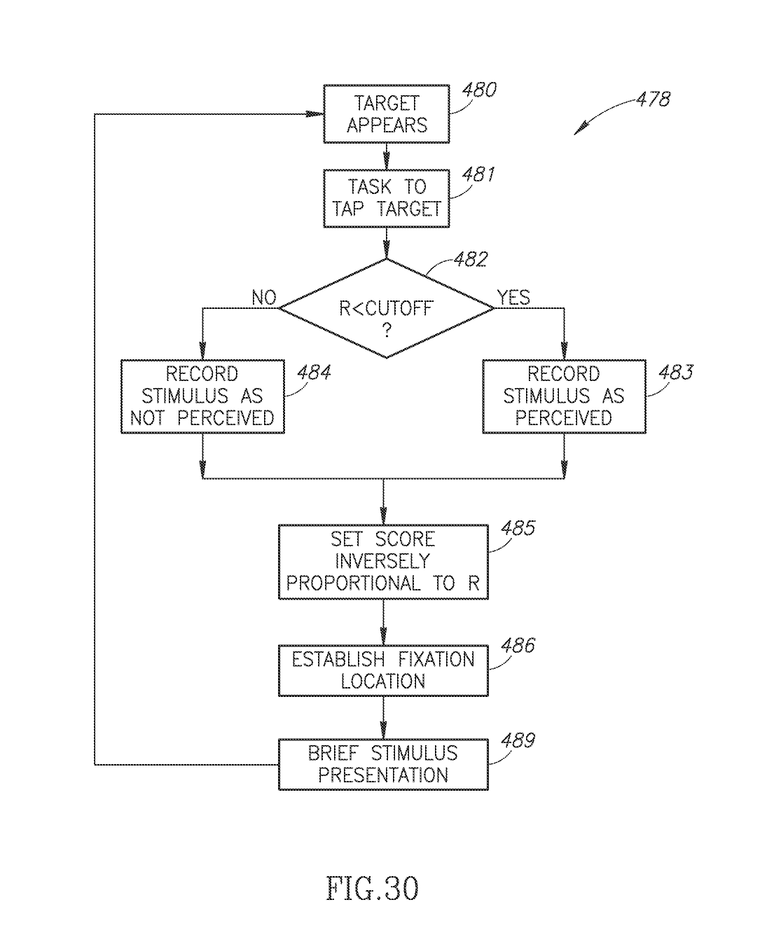

The speed tapping game cycle is represented in a flow chart 478 shown in FIG. 30. In step 486, a fixation location is established with a conspicuously visible symbol, such as a large blinking circle. Then, in step 489, a stimulus is presented briefly. After a brief delay T1, a target appears at the same place as the stimulus presented in step 480, and the player is tasked with tapping on the target in step 481. If the tap is on target and the reaction time R is less than a preset cutoff value in step 482, then the stimulus is recorded as perceived in step 483. Otherwise, the stimulus is recorded as not perceived in step 484. In step 485, the value of the score increment is inversely proportional to the reaction time R. That is, the faster the reaction, the greater the score acquired with the tap. The location of the target becomes the new fixation location in step 486. And the game cycle is repeated until the visual field is completely mapped according to FIGS. 11-13, as described above.

Various game scenarios could be used to make the visual field game more interesting when played repeatedly. One scenario could be a “whack a mole” game, where the circular stimuli and targets are made to resemble moles.

And if the player fails to whack (tap) the mole targets in time, the mole successfully steals carrots from the garden and the player loses points. Those skilled in the art will appreciate that other game scenarios may be used to provide the visual field game of the present invention.

### Advantages

Embodiments of the current invention are a video game-based VF test that solves many problems involved in adapting visual field testing from a large apparatus used in a controlled clinical environment to a small mobile device that could be used at home. Examples of a few of the problems addressed by some or all of the embodiments are discussed below.

Problem #1: The screen is too small.
Solution: Dynamic fixation increases the effective display area 4-fold.

The conventional perimeter uses a large spherical projection surface to cover a large range of visual angle. The surface area of a mobile computing device such as the iPad® is much smaller, and subtends a much smaller visual angle even with a relatively short working distance between the eye and the display screen. The present invention overcomes this problem by the use of dynamic fixation. In conventional perimetry, the fixation point is a fixed central point. Thus, the testable range of visual angle is measured from the center to the periphery. In the present invention, the fixation target location varies, and can be at the edge of the display area. Therefore, the testable range of visual angle is measured from edge to edge. This provides for a 4-fold increase of the effective visual angle test range given the same visual stimulus display area.

Problem #2: Ambient illumination is not standardized.
Solution: Use the video camera to sense ambient light.

In conventional VF testing, a technician dims the room light to a very low level once the subject is seated at the testing apparatus. The background illumination on the projection surface is then set to a standard level. In the present invention, the built-in video camera on the mobile computing device is used to sense the ambient light level and instruct the user to adjust room lighting to an acceptable level in the low scotopic range.

Problem #3: Working distance is not fixed.
Solution: Use video camera and occluder pattern of known size to establish the working distance.

In conventional VF testing, the subject's head is stabilized on a chin-forehead rest to fix the distance between the eye and visual stimuli to a preset distance. In the present invention, the working distance is monitored and adjusted by the video camera built into the mobile computing device. The camera captures images of an occluder worn over the eye not being tested. The occluder has a recognizable pattern of known dimension so that the working distance can be calculated by its apparent size in the video images. The device uses this information to instruct the subject to move the head to the correct working distance. Alternatively, the system scales the entire game according to the measured distance as described above. This feature is provided as an optional setup, which can be toggled on/off before starting a game by accessing the preference configuration pane.

### Other Advantages:

1) Embodiments of the current invention can be implemented on common consumer-owned hardware platforms such as a laptop computer or a tablet computer (i.e. the iPad® 2) or a video game playing station. This allows for more frequent repetitions of the VF test.
2) The embodiments of the game optimize the input devices available on the tablet computer—the touch screen and the video camera.
3) The subject's head is not constrained by a chin-forehead rest. This improves comfort.
4) Dynamic visual fixation points are more natural and less tiring compared to fixed central fixation points.
5) The subject is tasked to move a pointer towards the visual stimulus. This a more specific response compared to the clicker used in conventional visual field testing. The specificity reduces false positive responses. This also allows a faster pace of the game which helps to prevent boredom and hold attention.
6) As an alternative to manual control, head and eye tracking-based pointer control can speed up game play and VF testing.
7) The game uses interesting visual stimuli, visual action, and background scenery to help hold subject attention.
8) The game uses background music and action-generated sound to help hold subject attention.
9) The game keeps a score related to subject performance towards the game goal to help hold subject attention and to motivate repeated playing of the game.

10) The pace of the game is kept commensurate to player skill to help keep interest.

11) The video display of the game device can easily change color, pattern, and movement to capture different aspects of visual perception and to facilitate early detection of glaucoma.

### Example Hardware Environment

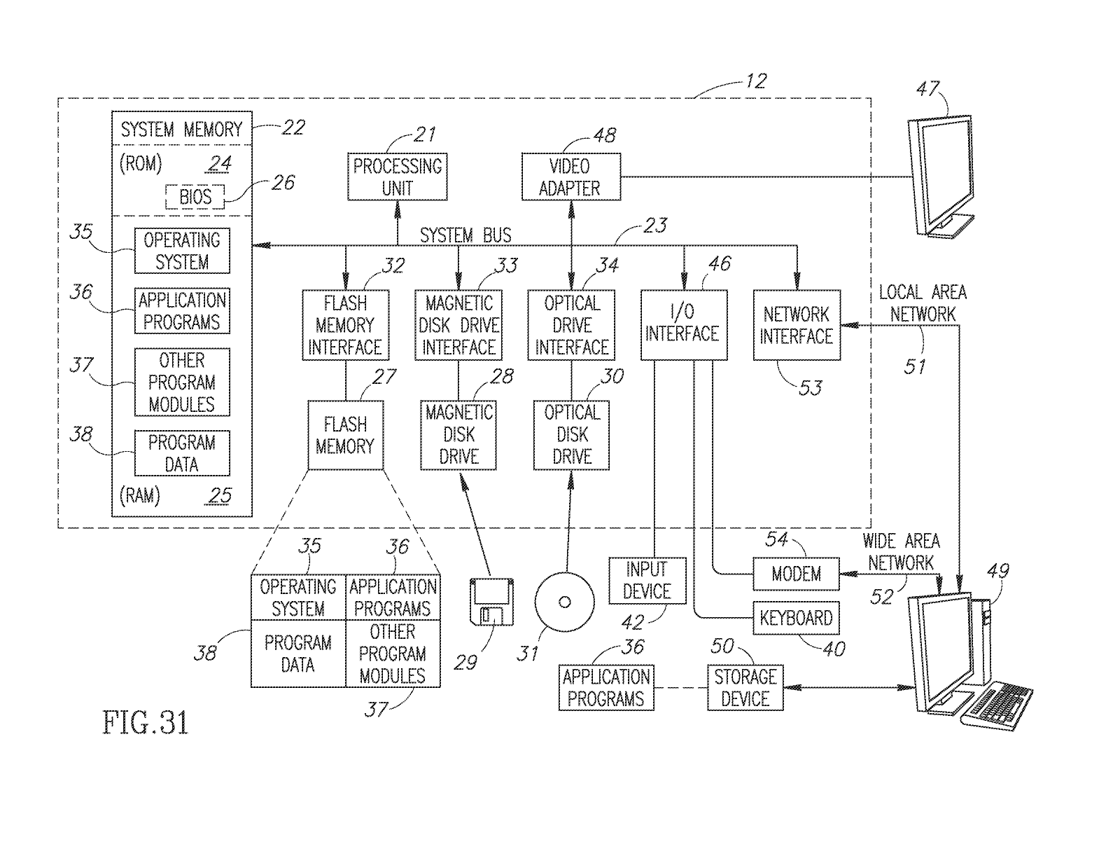

FIG. 31 is a diagram of hardware and an operating environment in conjunction with which implementations of the device 100 may be practiced. The description of FIG. 31 is intended to provide a brief, general description of suitable computer hardware and a suitable computing environment in which implementations may be practiced. Although not required, implementations are described in the general context of computer-executable instructions, such as program modules, being executed by a computer, such as a personal computer. Generally, program modules include routines, programs, objects, components, data structures, etc., that perform particular tasks or implement particular abstract data types.

Moreover, those skilled in the art will appreciate that implementations may be practiced with other computer system configurations, including hand-held devices, multiprocessor systems, microprocessor-based or programmable consumer electronics, network PCs, minicomputers, mainframe computers, tablet computers, smartphones, and the like. Implementations may also be practiced in distributed computing environments where tasks are performed by remote processing devices that are linked through a communications network. In a distributed computing environment, program modules may be located in both local and remote memory storage devices.

The exemplary hardware and operating environment of FIG. 31 includes a general-purpose computing device in the form of a computing device 12. The device 100 may be implemented using one or more computing devices like the computing device 12.

The computing device 12 includes a system memory 22, the processing unit 21, and a system bus 23 that operatively couples various system components, including the system memory 22, to the processing unit 21. There may be only one or there may be more than one processing unit 21, such that the processor of computing device 12 includes a single central-processing unit (“CPU”), or a plurality of processing units, commonly referred to as a parallel processing environment. When multiple processing units are used, the processing units may be heterogeneous. By way of a non-limiting example, such a heterogeneous processing environment may include a conventional CPU, a conventional graphics processing unit (“GPU”), a floating-point unit (“FPU”), combinations thereof, and the like. The computing device 12 may be a tablet computer, a smartphone, a conventional computer, a distributed computer, or any other type of computer.

The system bus 23 may be any of several types of bus structures including a memory bus or memory controller, a peripheral bus, and a local bus using any of a variety of bus architectures. The system memory 22 may also be referred to as simply the memory, and includes read only memory (ROM) 24 and random access memory (RAM) 25. A basic input/output system (BIOS) 26, containing the basic routines that help to transfer information between elements within the computing device 12, such as during start-up, is stored in ROM 24. The computing device 12 further includes a flash memory 27, a magnetic disk drive 28 for reading from or writing to a removable magnetic disk 29, and an optical disk drive 30 for reading from or writing to a removable optical disk 31 such as a CD ROM, DVD, or other optical media.

The flash memory 27, magnetic disk drive 28, and optical disk drive 30 are connected to the system bus 23 by a flash memory interface 32, a magnetic disk drive interface 33, and an optical disk drive interface 34, respectively. The drives and their associated computer-readable media provide nonvolatile storage of computer-readable instructions, data structures, program modules, and other data for the computing device 12. It should be appreciated by those skilled in the art that any type of computer-readable media which can store data that is accessible by a computer, such as magnetic cassettes, hard disk drives, solid state memory devices (“SSD”), USB drives, digital video disks, Bernoulli cartridges, random access memories (RAMs), read only memories (ROMs), and the like, may be used in the exemplary operating environment. As is apparent to those of ordinary skill in the art, the flash memory 27 and other forms of computer-readable media (e.g., the removable magnetic disk 29, the removable optical disk 31, flash memory cards, hard disk drives, SSD, USB drives, and the like) accessible by the processing unit 21 may be considered components of the system memory 22.

A number of program modules may be stored on the flash memory 27, magnetic disk 29, optical disk 31, ROM 24, or RAM 25, including an operating system 35, one or more application programs 36, other program modules 37, and program data 38. A user may enter commands and information into the computing device 12 through input devices such as a keyboard 40 and input device 42. The input device 42 may include touch sensitive devices (e.g., a stylus, touch pad, touch screen, or the like), a microphone, joystick, game pad, satellite dish, scanner, video camera, depth camera, or the like. In a preferred embodiment, the user enters information into the computing device using an input device 42 that comprises a touch screen, such as touch screens commonly found on tablet computers (e.g., an iPad® 2). These and other input devices are often connected to the processing unit 21 through an input/output (I/O) interface 46 that is coupled to the system bus 23, but may be connected by other types of interfaces, including a serial port, parallel port, game port, a universal serial bus (USB), or a wireless interface (e.g., a Bluetooth interface). A monitor 47 or other type of display device is also connected to the system bus 23 via an interface, such as a video adapter 48. In addition to the monitor, computers typically include other peripheral output devices (not shown), such as speakers, printers, and haptic devices that provide tactile and/or other types physical feedback (e.g., a force feedback game controller).

The computing device 12 may operate in a networked environment using logical connections (wired and/or wireless) to one or more remote computers, such as remote computer 49. These logical connections are achieved by a communication device coupled to or a part of the computing device 12 (as the local computer). Implementations are not limited to a particular type of communications device or interface.

The remote computer 49 may be another computer, a server, a router, a network PC, a client, a memory storage device, a peer device or other common network node or device, and typically includes some or all of the elements described above relative to the computing device 12. The remote computer 49 may be connected to a memory storage device 50. The logical connections depicted in FIG. 31 include a local-area network (LAN) 51 (wired or wireless) and a wide-area network (WAN) 52. Such networking environments are commonplace in offices, enterprise-wide computer networks, intranets and the Internet.

Those of ordinary skill in the art will appreciate that a LAN may be connected to a WAN via a modem using a carrier signal over a telephone network, cable network, cellular network (e.g., a mobile communications network such as 3G, 4G, etc.), or power lines. Such a modem may be connected to the computing device 12 by a network interface (e.g., a serial or other type of port). Further, many laptop or tablet computers may connect to a network via a cellular data modem.

When used in a LAN-networking environment, the computing device 12 may be connected to the local area network 51 through a network interface or adapter 53 (wired or wireless), which is one type of communications device. When used in a WAN networking environment, the computing device 12 typically includes a modem 54, a type of communications device, or any other type of communications device for establishing communications over the wide area network 52 (e.g., the Internet), such as one or more devices for implementing wireless radio technologies (e.g., GSM, etc.).

The modem 54, which may be internal or external, is connected to the system bus 23 via the I/O interface 46. The modem 54 may be configured to implement a wireless communications technology (e.g., mobile telecommunications system, etc.). In a networked environment, program modules depicted relative to the personal computing device 12, or portions thereof, may be stored in the remote computer 49 and/or the remote memory storage device 50. It is appreciated that the network connections shown are exemplary and other means of and communications devices or interfaces for establishing a communications link between the computers may be used.

The computing device 12 and related components have been presented herein by way of particular example and also by abstraction in order to facilitate a high-level view of the concepts disclosed. The actual technical design and implementation may vary based on particular implementation while maintaining the overall nature of the concepts disclosed.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. A computer-implemented method for visual field testing, comprising:
displaying a first fixation target on a display of a computing device at a first location;
displaying a first stimulus target briefly on the display at a second location spaced apart from the first location of the first fixation target;
monitoring for a first input from the user indicating a perception of the first stimulus target during a predetermined expected response time;
recording whether the user perceived the first stimulus target based on the presence or a characteristic of the first input received within the expected response time;
displaying a second stimulus target briefly on the display at a third location spaced apart from the second location of the second fixation target, wherein the second location is a second fixation target;
monitoring for a second input from the user indicating a perception of the second stimulus target during the predetermined expected response time;
recording whether the user perceived the second stimulus target based on the presence or a characteristic of the second input received within the expected response time; and
assessing the user's visual field based on the first second inputs of the user.

2. The computer-implemented method of claim 1, further comprising:
monitoring for a third input from the user indicating the execution of a task with respect to the first stimulus target;
recording whether the user completed the task based on the presence or a characteristic of the third input; and
displaying a score on the display of the computing device dependent on whether the user completed the task.

3. The computer-implemented method of claim 1, wherein monitoring for the first and second inputs comprises monitoring signals from a user input device comprising a touchscreen.

4. The computer-implemented method of claim 1, wherein monitoring for the first and second inputs comprises monitoring for at least one of: the user's head movements and the user's eye movements.

5. The computer-implemented method of claim 1, further comprising displaying numerous stimulus targets in succession at numerous locations on the display, wherein the location of each stimulus target becomes the location of an immediately subsequent fixation target.

6. The computer-implemented method of claim 5, further comprising generating a visual field map based on recorded perceptions of the plurality of stimulus targets by the user.

7. The computer-implemented method of claim 1, further comprising:
capturing an image of the user using an image capture device of the computing device; and
determining the distance between the display of the computing device and the user based on the captured image.

8. The computer-implemented method of claim 7, further comprising:
comparing the determined distance to a predetermined distance value; and
providing instructions to the user to either increase or decrease his or her distance from the display based on the comparison.

9. The computer-implemented method of claim 7, further comprising:
modifying a characteristic of the first and second stimulus targets based on the determined distance.

10. The computer-implemented method of claim 9, wherein modifying a characteristic of the first and second stimulus targets comprises modifying the size of the first and second stimulus targets.

11. The computer-implemented method of claim 9, wherein modifying a characteristic of the first and second stimulus targets comprises modifying the distance between the first and second stimulus targets.

12. The computer-implemented method of claim 7, wherein assessing the user's visual field is dependent on the determined distance.

13. The computer-implemented method of claim 1, wherein the computing device comprises a tablet computer and the first and second inputs are received via a user input device comprising a touch screen of the tablet computer.

14. The computer-implemented method of claim 1, further comprising:
measuring ambient light level; and
automatically adjusting a brightness level of the display dependent on the measured ambient light level.

15. The computer-implemented method of claim 1, further comprising:
measuring ambient light level; and
providing a notification instructing the user to adjust the ambient light level.

16. The computer-implemented method of claim 1, further comprising transmitting data relating to the user's visual field from the computing device to an external computing device.

17. The computer-implemented method of claim 16, further comprising storing the data on the external computing device, and analyzing the data to detect the presence of an eye condition.

18. The computer-implemented method of 17, further comprising sending a notification from the external computing device to a computing device over a network indicative of the detected eye condition.

19. The computer-implemented method of claim 1, further comprising:
displaying numerous stimulus targets in succession at numerous locations on the display, wherein the location of each stimulus target becomes the location of an immediately subsequent fixation target, and each stimulus target is displayed for the predetermined expected response time;
capturing images of the user using an image capture device of the computing device and, for each captured image, determining the distance between the display of the computing device and the user based on the captured image; and
generating a visual field map based on recorded perceptions of the plurality of stimulus targets by the user and the determined distances.

20. The computer-implemented method of claim 19, further comprising:
modifying the shape or size of the stimulus targets based on the determined distances.

21. The computer-implemented method of claim 1, further comprising measuring a reaction time of the user corresponding to the time required by the user to generate an input in response to the display of the first or second stimulus targets.

22. The computer-implemented method of claim 21, wherein assessing the user's visual field is dependent on the measured reaction time.

23. A computer-implemented method for visual field testing, comprising:
sequentially displaying a plurality of fixation targets and stimulus targets at numerous locations on a display of a computing device, wherein the location of each stimulus target becomes the location of an immediately subsequent fixation target;
subsequent to displaying each stimulus target, monitoring for an input from the user via a user input device of the computing device indicating a perception of the stimulus target, and recording whether the user perceived the stimulus target based on the presence or a characteristic of the input received;
during the displaying of the plurality of fixation targets and stimulus targets, monitoring the distance between the user and the computing device by capturing images using an image capturing device of the computing device and analyzing the captured images; and
assessing the user's visual field based on the inputs of the user.

24. The computer-implemented method of claim 23, further comprising modifying a characteristic of the stimulus targets based on the determined distance.

25. The computer-implemented method of claim 24, wherein the characteristic comprises the size of the stimulus targets.

26. The computer-implemented method of claim 24, wherein the characteristic comprises the distance between sequentially displayed stimulus targets.

27. The computer-implemented method of claim 23, wherein each stimulus target is displayed for a predetermined expected response time.

28. The computer-implemented method of claim 27, further comprising, prior to sequentially displaying the plurality of fixation targets and stimulus targets, determining the predetermined expected response time for the user by measuring one or more response times for the user.

29. A system for testing visual field, comprising:
a display;
a user input device;
a camera; and
a computer operatively coupled to the display, the camera, and the user input device, the computer configured to:
sequentially display a plurality of fixation targets and stimulus targets at numerous locations on the display, wherein the location of each stimulus target becomes the location of an immediately subsequent fixation target;
subsequent to displaying each stimulus target, monitor for an input from the user via the user input device of the computing device indicating a perception of the stimulus target, and record whether the user perceived the stimulus target based on the presence or a characteristic of the input received;
during the displaying of the plurality of fixation targets and stimulus targets, monitor the distance between the user and the computing device by capturing images using the camera and analyzing the captured images; and
assess the user's visual field based on the inputs of the user.

30. The system of claim 29, wherein the computer is further configured to monitor the ambient light level by capturing images with the camera, wherein the computer is configured to adjust the brightness of the display dependent on the monitored ambient light level.

31. The system of claim 29, wherein the computer is further configured to: monitor the ambient light level by capturing images with the camera, the computer being configured to display a message on the display providing instructions to the user to adjust the ambient light level of the environment.

32. The system of claim 29, further comprising:
a communications interface operatively coupled to the computer and configured to communicate with an external computer system using wired or wireless communication.

33. A non-transitory computer-readable medium encoded with computer executable instructions, which when executed, performs a method comprising:
sequentially displaying a plurality of fixation targets and stimulus targets at numerous locations on a display of a computing device, wherein the location of each stimulus target becomes the location of an immediately subsequent fixation target, each stimulus target being displayed for a predetermined expected response time;
subsequent to displaying each stimulus target, monitoring for an input from the user via a user input device of the computing device indicating a perception of the stimulus target during the expected response time, and recording whether the user perceived the stimulus target based on the presence or a characteristic of the input received;
during the displaying of the plurality of fixation targets and stimulus targets, monitoring the distance between the user and the computing device by capturing images using an image capturing device of the computing device and analyzing the captured images; and
assessing the user's visual field based on the inputs of the user.

34. The non-transitory computer-readable medium of claim 33, further comprising measuring a plurality of response times each corresponding to the time between the displaying of a stimulus target and the input from the user indicating a perception of the stimulus target, wherein assessing the user's visual field is dependent on the measured response times.

35. The non-transitory computer-readable medium of claim 34, further comprising generating a user score that is inversely proportional to the measured reaction times.
